(19) United States
(12) Patent Application Publication
(10) Pub. No.: US 2014/0120631 A1
(43) Pub. Date: May 1, 2014

(54) METHOD OF DETECTING PROTEIN AND DETECTING DEVICE

(71) Applicants: Hiroyuki Fujita, Tokyo (JP); Mehmet Cagatay Tarhan, Tokyo (JP); Ryuji Yokokawa, Tokyo (JP); Stanislav L. Karsten, Rancho Palos Verdes, CA (US)

(72) Inventors: Hiroyuki Fujita, Tokyo (JP); Mehmet Cagatay Tarhan, Tokyo (JP); Ryuji Yokokawa, Tokyo (JP); Stanislav L. Karsten, Rancho Palos Verdes, CA (US)

(73) Assignee: THE FOUNDATION FOR THE PROMOTIOM OF INDUSTRIAL SCIENCE, TOKYO (JP)

(21) Appl. No.: 14/123,665

(22) PCT Filed: Jun. 3, 2012
(86) PCT No.: PCT/IB2012/002009

§ 371 Date: Jan. 14, 2014

Related U.S. Application Data
(60) Provisional application No. 61/492,632, filed on Jun. 2, 2011.

(51) Int. Cl. G01N 33/566

(52) U.S. Cl. CPC G01N 33/566
USPC 436/501; 422/69

(57) ABSTRACT

A protein detection device and method. The device has a main channel in which is attached either a polar rail molecule or a biomolecular motor which moves on the rail molecule in a direction corresponding to the polarity of the rail molecule. A sub-channel crosses the main channel for receiving a sample including tau-protein. The method includes feeding the sample to the device and detecting a ratio of mutated tau-protein included in the sample, based on motion of the biomolecular motor on the rail molecule or on motion of the rail molecule moved by action of the biomolecular motor.

### TECHNICAL FIELD

The present invention relates to a method of detecting protein and a detection device used for the method.

### BACKGROUND ART

Conventionally, it has been known that kinesin, which is a biomolecular motor, moves on microtubules (a type of rail molecule) through hydrolysis of ATP (adenosine tri-phosphate) (see, for example, Patent Document 1). In this case, the microtubule is a cylindrical filament with a polymeric structure assembled from tubulin monomers, has a diameter of about 25 [nm] and a length of tens of micrometers and has polarity, then kinesin moves from the minus end of the microtubule toward the plus end thereof.

Tau-protein, MAP (microtubule associated protein) involved in microtubule assembly in vivo, especially is attached to the microtubule of cerebral nerve cell. Healthy tau-protein contributes to polymerization of tubulins and stabilization of the microtubule, and supports transport of material in cells (see, for example, Non-Patent Document 1). Though, degenerated tau-protein (or mutated tau), such as tau-protein perphosphoic or localized tau-protein, impedes movement of biomolecular motor in vivo.

In the field of brain science, neuropathology, etc. it has been known that mutated tau forms tangled tan or tangles in entorhinal area in brain and causes neurodegenerative disease, disturbance of memory, or other tauopathy, such as Alzheimer (see, for example, Non-Patent Document 2). Then, as technologies to detect the existence of protein on-chip, method of using surface plasmon and using immunoassey have been proposed (see, for example, Non-Patent Document 3).

### REFERENCES

Patent Document [1]; JP-A-2006-312211
Non-Patent Document [1]; P. Tolnay, “Tau protein pathology in Alzheimer's disease and related disorders”, Neuropathology and applied neurobiology; vol. 25(3), pp. 171-187 (1999)
Non-Patent Document [2]: T. Kimura et al., EMBO J., 26, 5143-5152, 2007
Non-Patent Document [3]: M. Vestergaard at al., Talanta, 74, 1038-1042, 2008

### DISCLOSURE OF INVENTION

### Problem to be Solved by the Invention

However, above-mentioned techniques are usable for detecting not existence of mutated tau, such as tau-protein perphosphoic or localized tau-protein, but just existence of tau-protein. Tauopathy is thought to be treated early through, for example, recovering function as healthy tau by utilizing phosphatase inhibitory agent, if being diagnosed at low rate stage of mutated tau.

An object of the present invention is to solve the above-mentioned problems in the conventional technologies and to provide a method of detecting protein and a detection device for the method, in which a relative motion of biomolecular motor on a rail molecule stabilized by paclitaxel is monitored so that the rate of mutated tau included in a sample is detected easily and surely.

### Means for Solving Problem

Accordingly, the present invention provides a method of detecting protein based on a relative motion of a rail molecule which has a polarity, and a biomolecular motor which moves on the rail molecule in a direction corresponding to the polarity of the rail molecule, the method comprising stabilizing the rail molecule with stabilizing agent; attaching either the rail molecule or the biomolecular motor onto a base; feeding a sample including tau-protein to the rail molecule; and detecting a ratio of mutated tau-protein included in the sample, based on motion of the biomolecular motor moving on the rail molecule or on motion of the rail molecule moved by action of the biomolecular motor.

In another method of detecting protein of the present invention, the ratio of mutated tau-protein included in the sample is detected, based on the fact that motion of the biomolecular motor on the rail molecule stabilized with stabilizing agent is disturbed by healthy tau-protein included in the sample and is not disturbed by mutated tau-protein.

In yet another method of detecting protein of the present invention, the stabilizing agent is paclitaxel or decetaxel.

In yet another method of detecting protein of the present invention, the motion comprises moving velocity, moving distance or density or number of the biomolecular motor moving on the rail molecule, or moving velocity, moving distance or density or number of the rail molecule moved by action of the biomolecular motor

In yet another method of detecting protein of the present invention, the biomolecular motor binding with a micro-sphere is fed to the rail molecule attached onto a base, so that the motion of the biomolecular motor moving on the rail molecule is judged based on motion of the micro-sphere.

In yet another method of detecting protein of the present invention, the rail molecule with fluorescence is fed to the biomolecular motor attached onto a base, so that the motion of the rail molecule moved by action of the biomolecular motor is judged based on motion of the rail molecule with fluorescence.

The present invention also provides a detection device used for a method of detecting protein based on a relative motion of a rail molecule which has a polarity, and a biomolecular motor which moves on the rail molecule in a direction corresponding to the polarity of the rail molecule, the detection device comprising a main channel having an upper surface of a base as a bottom surface where a rail molecule stabilized with stabilizing agent is attached; and a sub channel extending in a direction crossing the main channel and being fed with a sample including tau-protein, and wherein motion of the biomolecular motor moving on the rail molecule can be monitored.

In another detection device of the present invention, a plurality of sub channels are included so that effects of tau-protein included in a plurality of samples on motions of the biomolecular motors moving on the rail molecules can be monitored simultaneously.

In yet another detection device of the present invention, the biomolecular motor binding with a micro-sphere is fed in the main channel so that the motion of the biomolecular motor moving on the rail molecule is judged based on motion of the micro-sphere.

The present invention also provides a detection device used for a method of detecting protein based on a relative motion of a rail molecule which has a polarity, and a biomolecular motor which moves on the rail molecule in a direction corresponding to the polarity of the rail molecule, the detection device comprising a nano channel portion including a plurality of nano channels each of which has an upper surface of a base as a bottom surface where a biomolecular motor is attached; an inlet connected to one end of the nano channel portion so that the rail molecule stabilized with stabilizing agent is fed in the inlet; and an outlet connected to the other end of the nano channel portion so that the rail molecule fed in the inlet and carried out from the nano channel portion can be detected in the outlet.

In another detection device of the present invention, the rail molecule fed in the inlet with fluorescence so that intensity of fluorescence can be detected in the outlet.

### Effect of the Inventions

In the method of detecting protein of the present invention, the method comprising stabilizing the rail molecule with stabilizing agent; attaching either the rail molecule or the biomolecular motor onto a base; feeding a sample including tau-protein to the rail molecule; and detecting a ratio of mutated tau-protein included in the sample based on motion of the biomolecular motor moving on the rail molecule or on motion of the rail molecule moved by action of the biomolecular motor. Therefore, in-vitro, the rate of mutated tau-protein included in the sample can be detected easily and surely.

In the detection device of the present invention, the detection device comprising a main channel having an upper surface of a base as a bottom surface where a rail molecule stabilized with stabilizing agent is attached; and a sub channel extending in a direction crossing the main channel and being fed with a sample including tau-protein, and wherein motion of the biomolecular motor moving on the rail molecule can be monitored. Therefore, with a simple structure, in-vitro, the rate of mutated tau-protein included in the sample can be detected easily and surely.

In the detection device of the present invention, the detection device comprising a nano channel portion including a plurality of nano channels each of which has an upper surface of a base as a bottom surface where biomolecular motor is attached; an inlet connected to one end of the nano channel portion so that the rail molecule stabilized with paclitaxel is fed in the inlet; and an outlet connected to the other end of the nano channel portion so that the rail molecule fed in the inlet and carried out from the nano channel portion can be detected in the outlet. Therefore, with a simple structure, in-vitro, the rate of mutated tau-protein included in the sample can be detected easily and surely.

### BRIEF DESCRIPTION OF DRAWINGS

FIG. 1 is a set of views showing a method of detecting mutated tau using a first detecting device according to the first embodiment of the present invention.

FIG. 2 is a set of schematic views of a microtubule and kinesin according to the first embodiment of the present invention.

FIG. 3 is a set of schematic views of relationship between a microtubule and tau-protein in vivo according to the first embodiment of the present invention.

FIG. 4 is a schematic view of Bead Assay according to the first embodiment of the present invention.

FIG. 5 is a set of schematic views of a method of detecting mutated tau according to the first embodiment of the present invention.

FIG. 6 is a microphotograph showing beads according to the first embodiment of the present invention.

FIG. 7 is a set of views showing devices used in the experiments according to the first embodiment of the present invention.

FIG. 8 is a graph showing the effect of tau-protein on motion of kinesin according to the first embodiment of the present invention.

FIG. 9 is a first graph showing the effect of density of tau-protein on motion of kinesin according to the first embodiment of the present invention.

FIG. 10 is a graph showing the effect of incubation time on motion of kinesin according to the first embodiment of the present invention.

FIG. 11 is a graph showing the effect of incubation time on the number of molecules of kinesin according to the first embodiment of the present invention.

FIG. 12 is a graph showing the effect of microtubule on motion of kinesin according to the first embodiment of the present invention.

FIG. 13 is a second graph showing the effect of density tau-protein on motion of kinesin according to the first embodiment of the present invention.

FIG. 14 is a schematic view of Gliding Assay according to the second embodiment of the present invention.

FIG. 15 is a set of schematic views of a method of detecting mutated tau according to the second embodiment of the present invention.

FIG. 16 is a microphotograph showing microtubules according to the second embodiment of the present invention.

FIG. 17 is a set of views showing devices used in the experiments according to the second embodiment of the present invention.

FIG. 18 is a graph showing the effect of tan-protein on motion of kinesin according to the second embodiment of the present invention.

FIG. 19 is a graph comparing the motions of kinesin according to the first and second embodiments of the present invention.

FIG. 20 is a graph showing the effect of incubation time on motion of kinesin according to the second embodiment of the present invention.

### BEST MODES FOR CARRYING OUT THE INVENTION

An embodiment of the present invention will now be described in detail with reference to the drawings.

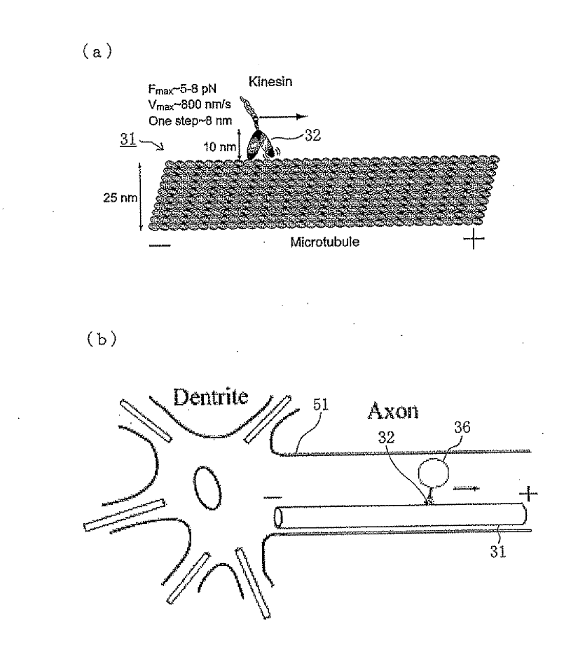

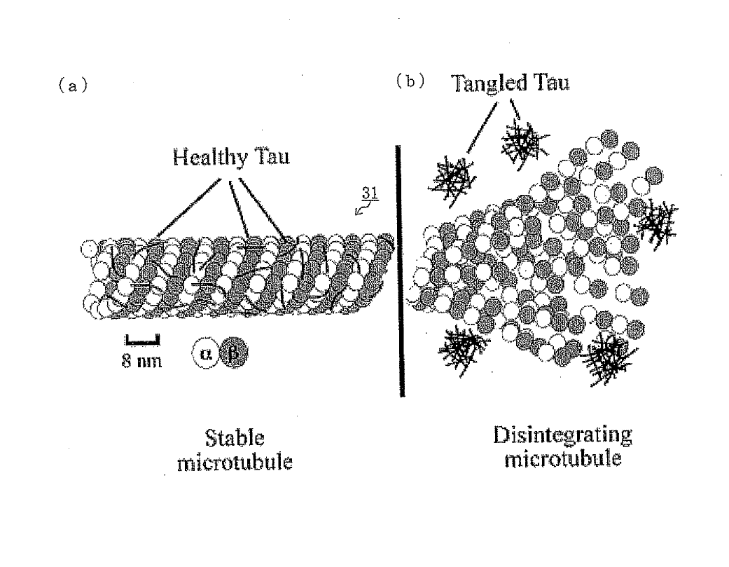

FIG. 2 is a set of schematic views of a microtubule and kinesin according to the first embodiment of the present invention. FIG. 3 is a set of schematic views of relationship between a microtubule and tau-protein in vivo according to the first embodiment of the present invention. In FIGS. 2, (a) shows just a microtubule and kinesin, (b) shows a microtubule and kinesin in a nerve cell, in FIGS. 3, (a) shows a stable microtubule and (b) shows a disintegrated microtubule.

In the respective figures, a reference numeral 31 designates a microtubule, a type of rail molecule in vivo, and a reference numeral 32 designates kinesin, a type of biomolecular motor which transfers in vivo an object material 36, such as an organelle, in a cell, such as a nerve cell 51.

A biomolecular motor, which is also called as a motor protein, binds to a cytoskeletal filament having polarity, and moves along the cytoskeletal filament in a predetermined direction. Several tens of biomolecular motors are present in cells, and the biomolecular motor employed in the present embodiment may be any bimolecular motor; for example, myosin or dynein. Since the present inventors conducted experiments employing, as a biomolecular motor, kinesin 32 as described hereinbelow, the embodiment will be described by taking, as an example, the case where kinesin 32 is employed as a biomolecular motor.

As described above, in cells, a cytoskeletal filament serves as a rail molecule for allowing a bimolecular motor to move. The cytoskeletal filament employed may be, for example, an actin filament. Since the present inventors conducted experiments employing microtubules 31 as rail molecules, the embodiment will be described by taking, as an example, the case where microtubules 31 are employed as rail molecules.

The microtubule 31 is one of three existing cytoskeletal filaments, and has a cylindrical filamentous structure (diameter: about 25 [nm], length: several tens of [μm]) obtained through polymerization of tubulin (i.e., a monomer). Tubulin is a heterodimer obtained through strong noncovalent binding between two globular polypeptides (i.e., α-tubulin and β-tubulin).

The microtubule 31 has polarity; one end thereof (the right end as viewed in FIG. 2) is a plus end, and the other end (the left end as viewed in FIG. 2) is a minus end. Such plus and minus ends are discriminated by the rate of polymerization of tubulin monomers (i.e., subunits) constituting the microtubule 31. Specifically, the end at which the rate of polymerization (extension) is high corresponds to a plus end, and the end at which the rate of polymerization (extension) is low corresponds to a minus end.

Kinesin 32 is a protein molecule having a full length of about 80 [nm] (size of a head portion: 10 [nm]). Kinesin 32, which has two globular head, portions and a twisted elongate coil portion, moves step by step on the microtubule 31 through alternate attachment and release of the head portions to and from the microtubule 31 in a repeated manner, as if both hands draw the filaments. In this case, kinesin 32 moves by steps (8 [nm] each) at a maximum speed of about 800 [nm/s]. As shown by the arrow of FIG. 2, kinesin 32 moves on the microtubule 31 from the minus end toward the plus end. Kinesin 32 generates a force of 5 to 8 [pN].

MAP called as tau-protein is attached to microtubule 31 in vivo, especially to microtubule 31 of cerebral nerve cell. As shown in FIG. 3(a), healthy tau-protein or healthy tau contributes to polymerization of tubulins and stabilization of microtubule 31. Though, as shown in FIG. 3(b), mutated tau-protein or mutated tau forms tangles and brakes away from microtubule 31 resulting in disintegration of microtubule 31.

Following synonyms must be noted.

tau protein=healthy tau

degenerated tau=protein assembled in the tangles (NFTs), partially digested or dysfunctional in any way

mutated tau=tau protein with mutation

hyperphosphorylated tau=tau protein which is excessively phosphorylated and not capable of binding to MTs

localized tau=tau protein in the known lacation

tangled tau=tau assembled in tangles such as NFTs

Next will be described a method for detecting mutated tau according to this embodiment.

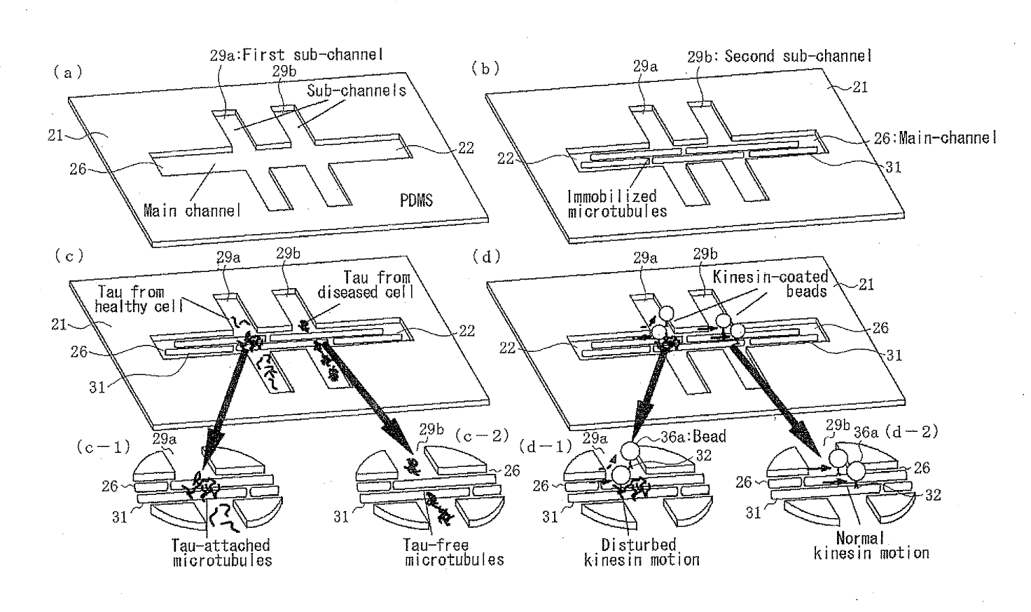

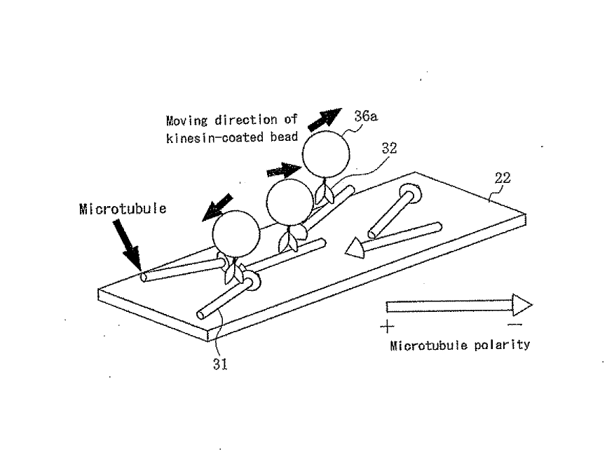

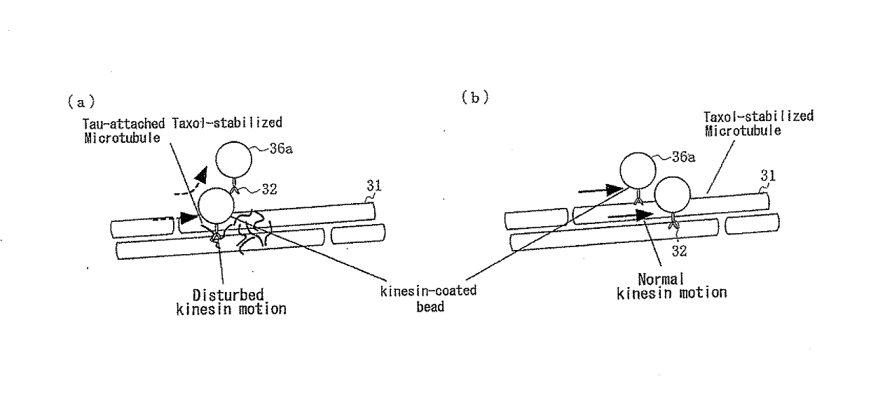

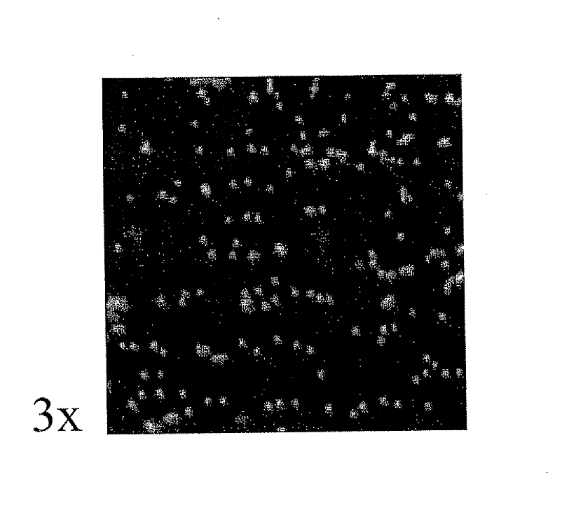

FIG. 1 is a set of views showing a method of detecting mutated tau using a first detecting device according to the first embodiment of the present invention. FIG. 4 is a schematic view of Bead Assay according to the first embodiment of the present invention. FIG. 5 is a set of schematic views of a method of detecting mutated tau according to the first embodiment of the present invention. FIG. 6 is a microphotograph showing beads according to the first embodiment of the present invention. In FIGS. 1, (a)-(d) each shows each step respectively, (c-1), (c-2), (d-1) and (d-2) show enlarged parts, in FIGS. 5, (a) shows a disturbed kinesin motion and (b) shows a normal kinesin motion.

The present inventors have invented a first and a second detection devices after-mentioned as detection devices to detect protein in-vitro, and a first and a second methods of detecting protein with utilizing the first and the second detection devices.

In the present embodiment, the first method of detecting protein will be described by taking, as an example, the case where mutated tau is employed as the protein to be detected by the method. Specific ally, the method is a method called as “Bead Assay” or “in-vitro Bead Assay” to detect protein by observing a bead 86a bound to protein, as shown in FIG. 4, where existence and rate of mutated tau included in a sample is judged by monitoring motion of kinesin 32, with the bead 36a bound thereto, along the microtubule 31 fixed on a base 22 such as a plate of glass.

In this case, the microtubules 31 are stabilized with stabilizing agent. Taxane compound, such as paclitaxel or docetaxel, can be used as stabilizing agent. Examples using paclitaxel will be described. That is, the microtubules 31 are “taxol-stabilized microtubules”, which are stabilized with paclitaxel. These microtubules 31 do not include any MAP such as tau-protein and polymerization of tubulins therein is kept stably with paclitaxel. Specifically, tubulin purified from porcine brains was polymerized into microtubules 31 in BRB 80 buffer (80 [mM] PIPES-NaOH pH=6.8, 1 [mM] MgCl2 1 [mM] EGTA) containing MgSO4 (1 [mM]) and GTP (1 [mM]). After incubating at 37[° C.] for 30 [min], microtubules 31 were stabilized with paclitaxel (40 [μm]) and diluted 100-fold in BRB 80 containing paclitaxel (20[μm]). For some experiments, microtubules 31 were diluted in BRB 80 solution containing both tau-protein and paclitaxel.

The present embodiment, like the invention described in Patent Document 1, employs a method for fixing microtubules 31 by kinesin 32 through inactivation of kinesin 32 by irradiation with light having a predetermined wavelength. In this method, firstly, numerous molecules of kinesin 32 are fixed onto the surface of a base 22. When a microtubule 31 is applied thereto, followed by supply of ATP, the microtubule 31 moves by motion of kinesin 32 serving as a biomolecular motor. Specifically, molecules of kinesin 32 fixed onto the surface of the base 22 move on the microtubule 31, and thus the microtubule 31 moves above the surface of the base 22. When the microtubule 31 reaches a predetermined position, kinesin 32 is exposed to light having a predetermined wavelength. Through this procedure, kinesin 32 is inactivated, and motion of kinesin 32 is stopped, with the microtubule 31 being attached to the head portions of kinesin 32. Therefore, the microtubule 31 stops moving. In this case, since the head portions of kinesin 32 remain attached to the microtubule 31, the microtubule 31 is fixed.

Beads 36a coated with kinesin 32 were used as object materials 36 for monitoring the motion of kinesin 32. Specifically, biotinylated kinesin (GST-410-BCCP kinesin having a biotin-dependent enzyme called biotin carboxyl carrier protein (BOOM was used in the experiment. Biotinylated kinesin was expressed in E. coli. After culturing and purification, resulting biotinylated kinesin had a concentration of 0.07 [mg/ml].

Beads 36a were commercially available streptavidin-coated micro-spheres (CP01N/6905, Bangs Laboratories Inc.) with a diameter of 0.49 [μm]. Specific avidin-biotin binding was used to attach molecules of kinesin 32 to beads 36a.

And two types of recombinant tau-protein (Tau-441 human 2N4R) were used. One of those was healthy tau (T0576, Sigma) and the other one was mutated tau (P301L, rPeptide).

As mentioned before, the microtubules 31 stabilized with paclitaxel do not include any MAP. Therefore, in the case tau-protein is attached to the microtubules 31 stabilized with paclitaxel, as shown in FIG. 5(a), motion of kinesin 32 attached thereon is supposed to be disturbed, resulting in slow motion of kinesein 32 or dropping of molecules of kinesein 32 from the microtubules 31. On the other hand, in the case tau-protein is not attached to the microtubules 31 stabilized with paclitaxel, as shown in FIG. 5(b), motion of kinesin 82 thereon is supposed to be undisturbed, resulting in normal motion of kinesin 32 or rare dropping of molecules of kinesin 32 from the microtubules 31. In other words, it is supposed that, in-vitro, differences would be found in moving distance and moving velocity of molecules of kinesin 32 moving on the microtubules 31 and in density (or number) of molecules of kinesin 32 existing on the microtubules 31, according to whether tau-protein is attached or not to the microtubules 31 stabilized with paclitaxel.

On the other hand, healthy tau is supposed to attach to the microtubules 31 stabilized with paclitaxel, while mutated and tangle forming tau is not supposed to attached thereto. In other words, when feeding healthy tau to the microtubules 31 stabilized with paclitaxel, motion of kinesin 32, that is, moving velocity, moving distance and density (or number) of molecules of kinesin 32 would be affected, as shown in FIG. 5(a), though, when feeding mutated tau to the microtubules 31 stabilized with paclitaxel, motion of kinesin 32 would not be affected, as shown in FIG. 5(b).

Therefore, by feeding a sample including tau-protein purified from a test subject to the microtubules 31 stabilized with paclitaxel and by monitoring motion of kinesin 32, it would be possible to judge whether the tau-protein included in the sample is healthy or mutated based on the motion of kinesin 32, and, further, to detect the rate of mutated tau.

Based on such a view, the present inventors have invented a method of detecting degenerated or mutated tau-protein. In this case, mutated tau can be sensed or detected by using a detecting device as shown in FIG. 1.

In the respective figures, a reference numeral 21 designates a PDMS (Poly-dimethylsiloxane) film, where formed are a single main channel 26 extending straight and a pair of sub channels 29 extending in a direction crossing (in FIG. 1, at right angles) the main channel 26. One of the sub channels 29 is designated as a first sub channels 29a and the other is designated as a second sub channels 29b. Then, the base 22, served as glass slide, is attached to the lower surface of the PDMS film 21 and the upper surface of the base 22 functions as the bottom surface of the main channel 26 and the sub channels 29.

As shown in FIG. 1(b), microtubules 31, which are stabilized with paclitaxel, are immobilized and fixed onto the bottom surface of the main channel 26. Then solution of tau-protein is injected into the sub channels 29. In this case, as shown in FIG. 1(c), the solution of healthy tau is injected into is injected into the first sub channels 29a and the solution of mutated tau is injected into the second sub channels 29b. As a result, healthy tau is attached to the microtubules 31, as shown in FIG. 1(c-1), though mutated tau is not attached to the microtubules 31; as shown in FIG. 1(c-2).

Then, as shown in FIG. 1(d), beads 36a coated with kinesin 32 are fed into the main channel 26, followed by activation of kinesin 32. With bead 36a, kinesin 32 moves on the microtubule 31, though its motion is disturbed at a part where healthy tau attached, resulting in slow motion of kinesin 32 or dropping of molecules of kinesin 32 from the microtubule 31, as shown in FIG. 1(d-1). On the other hand, as its motion is not disturbed at a part where no tau-protein attached, with bead 36a, kinesin 32 moves on the microtubule 31 smoothly without dropping therefrom, as shown in FIG. 1(d-2).

FIG. 6 is a microphotograph taken by the present inventors, showing beads 36a coated with kinesin 32.

Thus, by monitoring and recording motion of kinesin. 32 both at one part where healthy tau fed and at another part where mutated tau fed on the microtubule 31 in advance, then by comparing motion of kinesin 32 at a part where a sample including tau-protein purified from a test subject is fed, with the recorded result, it is possible to judge whether mutated tau is included or not in the sample and, also, it is possible to judge the rate of mutated tau, if any, in the sample.

Next will be described methods and structures of devices used in the experiments the present inventors conducted.

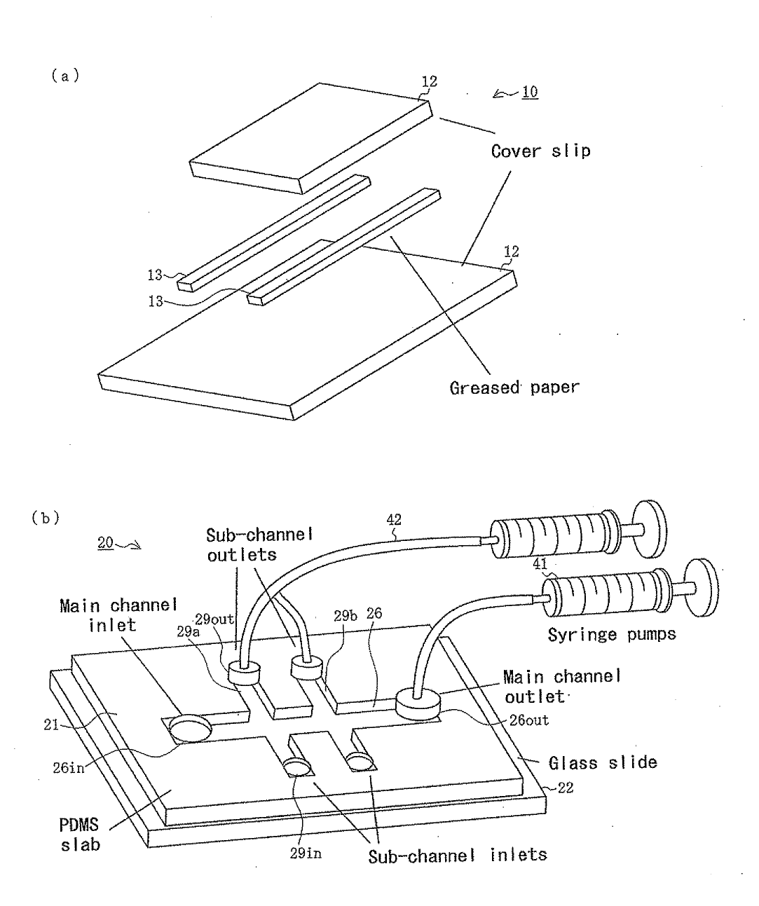

FIG. 7 is a set of views showing devices used in the experiments according to the first embodiment of the present invention. In FIGS. 7, (a) shows a schematic view of flow cell and (b) shows a schematic view of the first detecting device.

The present inventors conducted basal experiments using a flow cell 10 as shown in FIG. 7(a). The flow cell 10 includes a pair of cover slips 12 made of transparent plates such as glass plates, and a pair of spacer members 13 for keeping space between the cover slips 12. The spacer members 13 are made of greased paperboard, etc. The microtubules 31 and beads 36a coated with kinesin 32 were fed into the space defined by the pair of cover slips 12 and a pair of spacer members 13, and motion of kinesin 32 on the microtubules 31 was monitored.

A first chip 20 as a micro-fluidics chip, as shown in FIG. 7(b), was finally adopted as the first detecting device for judging whether mutated tau was included or not in the sample and for judging the rate of mutated tau, if any, in the sample. The first chip 20 includes the base 22, as a glass slide, and the PDMS film 21 attached onto the base 22, and the PDMS film 21 includes, as mentioned before, one main channel 26 extending straight and a pair of sub channels 29 extending in a direction crossing the main channel 26 at right angles. One of the sub channels 29 is designated as a first sub channels 29a and the other is designated as a second sub channels 29b. Then, the base 22 is attached to the lower surface of the PDMS film 21 and the upper surface of the base 22 functions as the bottom surface of the main channel 26 and the sub channels 29.

The PDMS film 21 was produced through photo-lithography technique, like the invention described in Patent Document 1. Using resist, such as SU-8 50 (MicroChem), convex patterns with a height of about 50 [μm] corresponding to the main channel 26 and the sub channels 29 were formed on a top surface of silicon wafer (not illustrated). Then, a prepolymer of PDMS, such as Silgard 184 (Dow Corning) was applied so as to cover the top surface of silicon wafer, and the prepolymer was cured, followed by removal of the thus-cured prepolymer. Through this procedure, the PDMS film 21 shown in FIG. 7(b) was formed.

Finally, the PDMS film 21 was exposed to oxygen plasma (50 [sccm], 20 [Pa], 65 [W], 8 [sec]), then was bound so as to adhere closely to the upper surface of the base 22.

In the first chip 20 the present inventors produced, the depth, width and length of the main channel 26 were 50 [μm], 200 [μm] and 20 [mm] respectively, and those of each sub channel 29 were 50 [μm], 100 [μm] and 15 [mm] respectively.

Further, the present inventors used an inverted microscope (Olympus IX-71) with a DIC (Differential Interference Contrast) setup for monitoring experiments. Images were recorded as experimental records using a camera (Photometrics Cascade 512II) and were processed using software such as MetaMorph and Cosmos.

The flow cell 10 was held directly on the stage of the inverted microscope. As shown in FIG. 7(b), the first chip 20 was integrated with a pair of syringe pump 41 (Kd Scientific). The syringe pumps 41 were connected through connection pipes 42 to an outlet 26out of the main channel 26 and outlets 29out of the sub channels 29 respectively and were used for sacking out fluid from the main channel 26 and the sub channels 29. In FIG. 7(b), reference numerals 26 in and 29 in designate inlets of the main channel 26 and the sub channels 29 respectively.

To detect the activity of kinesin 32, its motion along the microtubules 31 had to be monitored. As mentioned before, using beads 36a coated with kinesin 32 made it possible to use DIC setup for measuring the motion, typically the average velocity, of kinesin 32 along the immobilizes microtubules 31.

In the experiments, the cover slip 12 and the base 22, made of glass plates coated with poly-L-lysine (PLL, a polycation that can be bind to any negatively charged proteins), were used for immobilizing the microtubules 31. At this point, two different ways were taken to prepare the tau-attached microtubules 31.

The first way was to prepare tau-attached microtubules 31 (TMT: microtubules 31 with healthy tau or MuTMT; microtubules 31 with mutated tau) separately. Microtubules 31, diluted 100-fold in BRB 80 solution containing healthy tau (or mutated tau), were incubated inside a tube at 37[° C.] for 20 [min]. Then, tau-attached microtubules 31 were injected into the PLL-coated flow cell 10 and incubated for 3 [min]. After flowing beads 36a coated with kinesin 32, 1 [mM] ATP solution was flushed to activate kinesin 32.

The second way was based on on-site attachment. In this case, microtubules 31, diluted 100-fold in BRB 80 solution, were first incubated for 3 [min] for immobilization inside the PLL-coated flow cell 10. Then, tau solution was injected for attachment to microtubules 31. Experiments with different incubation time for tau attachment were conducted as mentioned later. Resulting immobilized TMTs were used for monitoring motion of beads 86a coated with kinesin 32 in presence of 1 [mM] ATP solution.

Experiments based on the second way (on-site attachment) were conducted using the first detection device as shown in FIG. 1. The main channel 26 was first coated with PLL and then microtubules 31 were immobilized. Tau solution was injected into one of the sub channels 29 while BRB 80 solution was injected into the other sub channel 29 as a control experiment. After incubating for 25 [min], the beads 36a coated with kinesin 32 were flown into the main channel 26. Finally, 1 [mM] ATP solution was flushed to activate kinesin 32.

Next will be described details and results of the experiments the present inventors conducted.

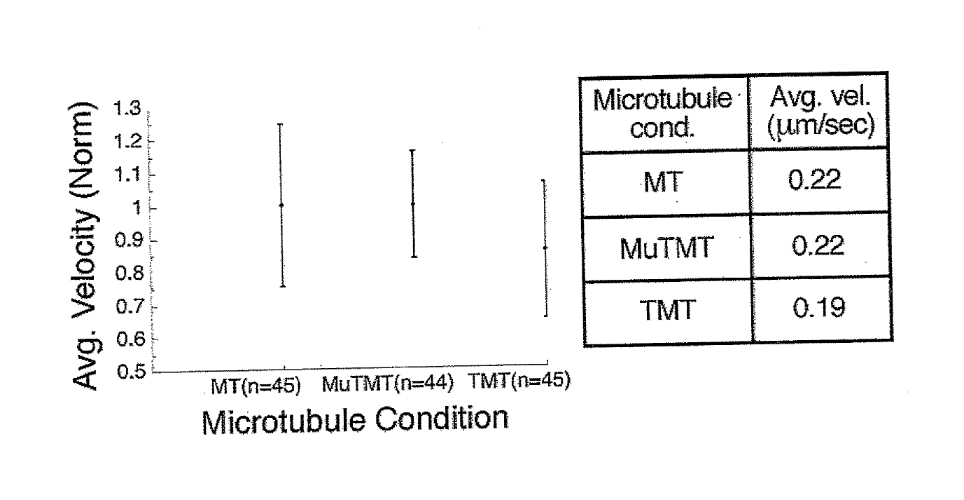

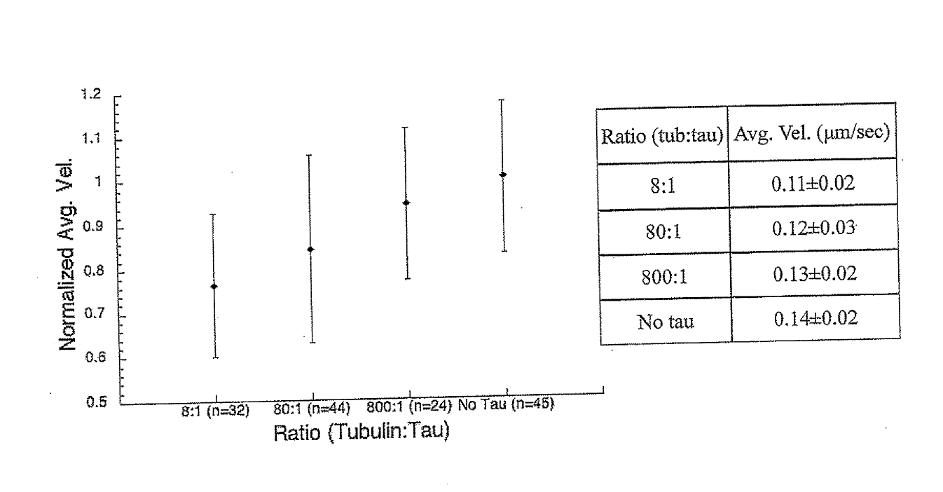

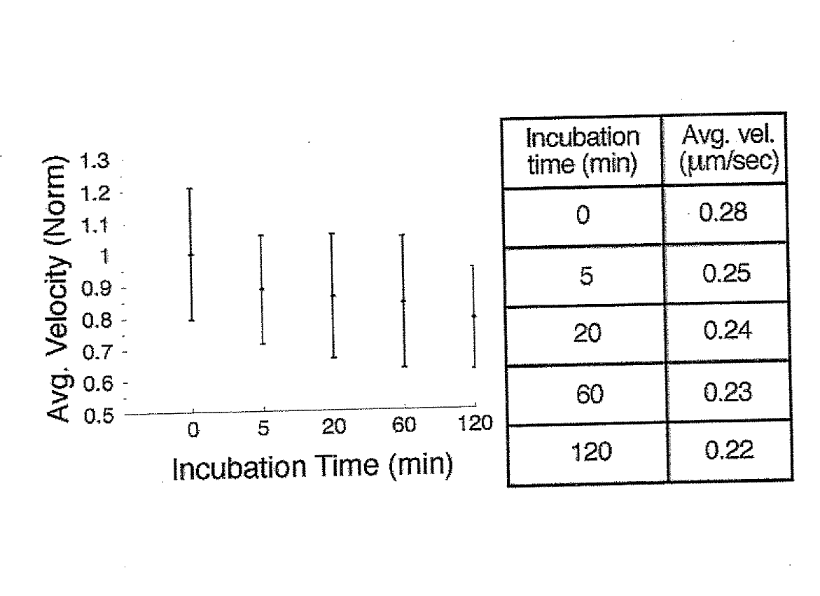

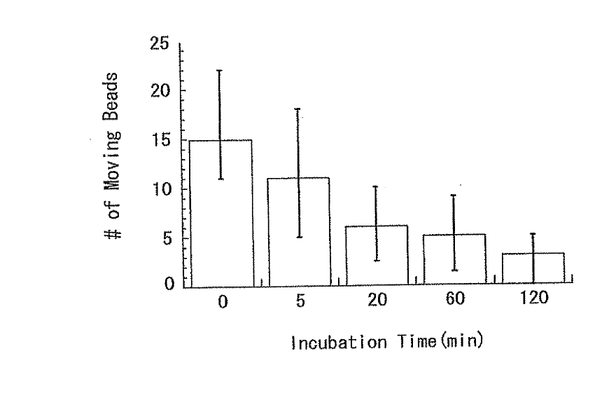

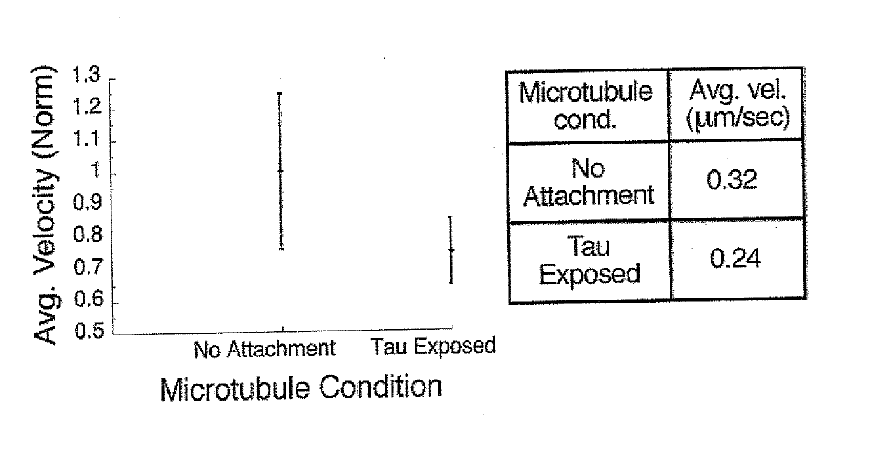

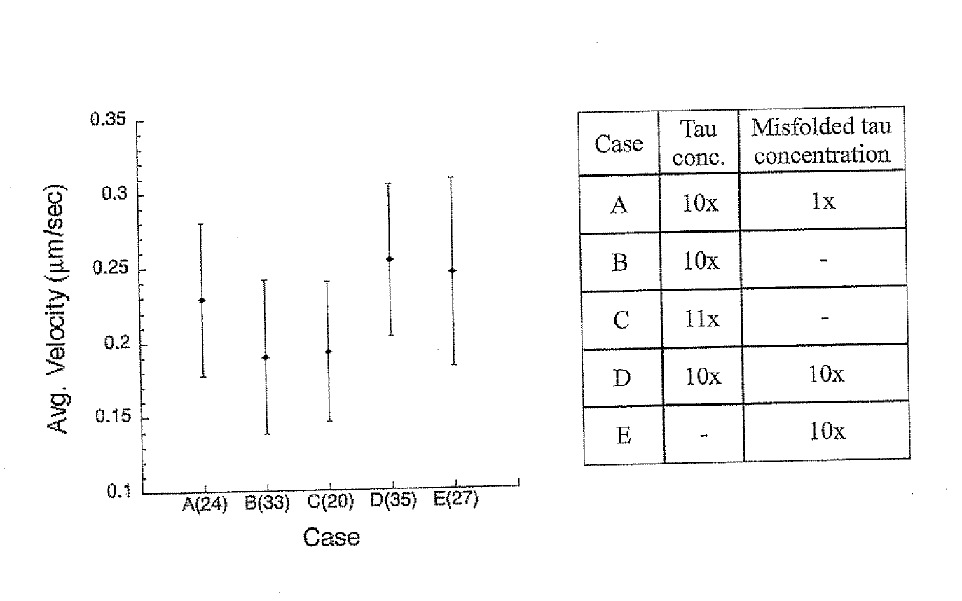

FIG. 8 is a graph showing the effect of tau-protein on motion of kinesin according to the first embodiment of the present invention. FIG. 9 is a first graph showing the effect of density of tau-protein on motion of kinesin according to the first embodiment of the present invention. FIG. 10 is a graph showing the effect of incubation time on motion of kinesin according to the first embodiment of the present invention. FIG. 11 is a graph showing the effect of incubation time on the number of molecules of kinesin according to the first embodiment of the present invention. FIG. 12 is a graph showing the effect of microtubule on motion of kinesin according to the first embodiment of the present invention. FIG. 13 is a second graph showing the effect of density tau-protein on motion of kinesin according to the first embodiment of the present invention.

Experiments were conducted under three different conditions.

The first condition, corresponding to the first way, was using pre-attachment of tau before immobilizing the microtubules 31 in the flow cell 10. Microtubules 31, diluted 100-fold in BRB 80 solution containing healthy tau (or mutated tau) with a ratio of 40:1 (tubulin monomer:tau molecule), were incubated inside a tube at 37[° C.] for 20 [min]. Meanwhile, as a control experiment, normal microtubules 31 were diluted 100-fold in BRB 80 solution and incubated inside a tube at 37[° C.] for 20 [min]. After applying the assay protocol, beads 36a coated with kinesin 32 were monitored and the average velocity was measured for each case.

The average velocities of beads 36a along MTs and MuTMTs were measured as 0.22±0.06 [μm/sec] (n=45) and 0.22±0.04 [μm/sec] (n=44) respectively. The letter n means the number of beads 36a monitored. On the other hand, the average velocities of beads 36a along TMT was measured as 0.19±0.05 [μm/sec] (n=45).

As shown in FIG. 8, the meaning of the experiments would be clear when comparing their results with the result of the control experiment. FIG. 8 shows comparison of normalized average velocities of beads 36a coated with kinesin 32 moving along normal microtubules 31 (MTs) with different types of tau attached microtubules 31 (MT: normal MTs, MuTMT: mutated tau attached MTs, and TMT: healthy tau attached MTs) in the flow cell 10. Error bars in FIG. 8 correspond to standard deviation.

The result of the Student's t test (see, for example, Non-Patent Document 4) for MuTMT and TMT cases are p=0.86 and p<0.01 respectively.
Non-Patent Document [4]: S. M. Dunn, A. Constantinides, P. V. Moghe, “Numerical Methods in Biomedical Engineering”, Elsevier Academic Press, 2006

Also experiments concerning the effect of density of tau-protein on the average velocities of beads 36a were conducted. FIG. 9 shows difference of normalized average velocities of beads 36a coated with kinesin 32 corresponding to different ratios of microtubules 31 and tau-protein in the flow cell 10. As shown in FIG. 9, in cases where ratios of tubulin monomer:tau molecule are 8:1, 80:1 and 800:1, normalized average velocities of beads 36a along tau attached microtubules 31 were 0.11±0.02 [μm/sec] (n=32), 0.12±0.03 [μm/sec] (n=44), 0.13±0.02 [μm/sec] (n=24) and 0.14±0.02 [μm/sec] (n=45) respectively. The results showed a decrease in average velocities of beads 36a with an increase of tan-protein attached onto microtubules 31.

The second experimental condition, corresponding to the second way, was using on-site attachment of tau-protein on microtubules 31 in the flow cell 10. For this experiment, 1 [μg/ml] tau-protein solution was injected in the flow cell 10 after immobilizing microtubules 31. Average velocities of beads 36a coated with kinesin 32 were measured for different incubation times (0, 5, 20, 60 and 120 [min]).

As shown in FIG. 10, the result showed a decrease in average velocities of beads 36a with increasing incubation time. FIG. 10 shows normalized average velocities of beads 36a coated with kinesin 32 according to different incubation times in case where tau-protein was attached on-site onto microtubules 31 in the flow cell 10. The numbers of beads 36a monitored were n=65, 44, 25, 20 and 8 respectively. In FIG. 10, error bars correspond to standard deviation and X-axis is not scaled.

Furthermore, FIG. 11 shows the average number of moving beads 36a per observation. According to this figure, the effect of the on-site attachment of tau-protein to immobilized MTs in the flow cell 10 became apparent. In FIG. 11, error bars correspond to standard deviation and X-axis is not scaled.

The number of moving beads 36a was more than 16/observation for control experiment (incubation time, t=0 [min]). However, with increasing incubation time the number of moving beads 36a decreased down to 2/observation for the longest incubation time (t=120 [min]). 4 observations were done for each case of incubation time.

The third experimental condition, corresponding to the second way, was using on-site attachment of tau-protein on microtubules 31 in the first detecting device as shown in FIG. 1. As mentioned before, the main channel 26 was first coated with PLL and than microtubules 31 were immobilized. 1 [μg/ml] tau-protein solution was injected into one of the sub channels 29 and incubated for 25 [min], while BRB 80 solution was injected into the other sub channel 29 as a control experiment. The preliminary result is shown in FIG. 12. Average velocities of beads 36a coated with kinesin 32 on MTs and TMTs were measured as 32±0.08 [μm/sec] (n=15) and 0.24±0.03 [μm/sec] (n=9) respectively.

FIG. 12 shows comparison of the normalized average velocities of beads 36a coated with kinesin 32 moving at different crossing of the main channel 26 and sub channels 29. The numbers of monitored beads 36a were n=15 and 9 respectively. A significant decrease was seen for the crossing exposed to the tau-protein solution when compared to the crossing exposed only to BRB 80 solution. Error bars in FIG. 12 correspond to standard deviation.

Even though it is known that tau-protein has an important role on stabilization of microtubules 31, the experiments showed that tau-protein had a negative effect on motion of kinesin 32. This was because stabilization of microtubules 31 had already been achieved by using paclitaxel. As a result, tau-protein was not useful for stabilization of microtubules 31 but rather acted as obstacles for kinesin 32. This idea comforts the result of experiments shown in FIG. 8.

Healthy tau-protein attaches successfully to microtubules 31. On the contrary, mutated tau-protein tends to bind each other to form tangles. These tangles have reduced capability of binding to microtubules 31 (see, for example, Non-Patent Document 1). As a result, a decrease on average velocities of beads 36a coated with kinesin 32 is expected when TMTs, microtubules 31 fed with healthy tau-protein, are used. Furthermore, as there will not be any tau attachment on microtubules 31, any difference is not expected on the average velocities of beads 36a coated with kinesin 32 between MuTMT case and MT case.

Using the before mentioned device as a molecular detector requires capability of providing information about level of attachment as well. FIG. 10 shows that longer incubation time increases the amount of tau-protein attached onto microtubules 31 resulting in decrease in the average velocities of beads 36a coated with kinesin 32. Furthermore, higher amount of tau-protein attachment hinders motion of kinesin 32 (and probably attachment of kinesin 32 onto microtubules 31 as well) resulting in easy detachment of beads 36a coated with kinesin 32. Thus, as shown in FIG. 11, the number of beads 36a coated with kinesin 32 moving along TMTs was decreased with increasing incubation time.

The present inventors also conducted experiments concerning the effect of density of mutated tau on the average velocities of beads 36a coated with kinesin 32. FIG. 13 shows comparison of normalized average velocities of beads 36a coated with kinesin 32 in the flow cell 10 with different densities of health tau and tangled tau. In cases of A-E, as shown in FIG. 13, the numbers of healthy tau-protein in comparison with microtubule 31, the numbers of mutated and tangled tau-protein in comparison with microtubule 31 and the value of n are {10 times, 1 time, n=24}, {10 times, 0, n=33}, {11 times, 0, n=20}, {10 times, 10 times, n=35}, and {0, 1.0 times, n=27} respectively. Thus, the average velocities of beads 36a coated with kinesin 32 were higher when using MuTMT than when using TMT.

Using the first chip 20 as shown in FIG. 7(b), capable of investigating different conditions, is crucial to minimizes the errors caused by the experimental setup. Experiments using the flow cell 10 require different device for each experimental case. On the other hand, the first chip 20 provides an excellent tool to conduct experiments under exactly the same conditions. This is because molecules of tau-protein attach on different fragments of the same microtubules 31 at different areas of the main channel 26. Moreover, the same beads 36a coated with kinesin 32 move in the main channel 26 for all different cases. Providing the same conditions for comparing different cases is an important advantage of the device for consistent results.

As described above, in the present embodiment, microtubules 31 stabilized with paclitaxel are attached on a base 22, a sample including tau-protein is fed to the microtubules 31, and a ratio of mutated tau included in the sample is detected based on motion of kinesin 32 moving on the microtubules 31.

And, a first chip 20 includes a main channel 26 with, as a bottom, a surface of the base 22 on which microtubules 31 stabilized with paclitaxel are attached, and sub channels 29 extending in a direction crossing the main channel 26 and the sample including tau-protein is fed therein, wherein motion of kinesin 82 moving on the microtubules 31 can be monitored.

Therefore, in-vitro, the rate of mutated tau included in the sample can be detected easily and surely. Then it would be possible to diagnose tauopathies, typified by Alzheimer, at the stage with low rate of mutated tau, and to cure them early by recovering functions of healthy tau utilizing phosphatase inhibitory agent etc.

Next, a second embodiment of the present invention will now be described. Structural features similar to the first embodiment are denoted by common reference materials, and repeated description of operation and effects similar to those of the first embodiment is omitted.

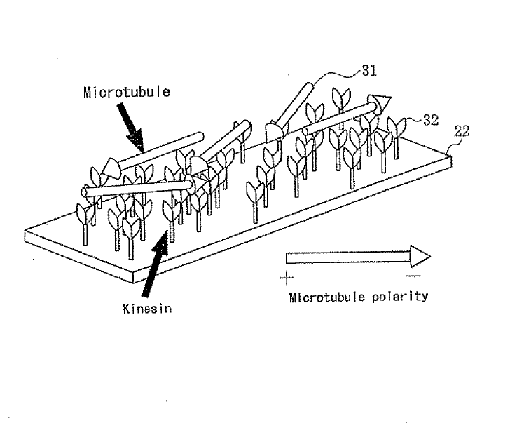

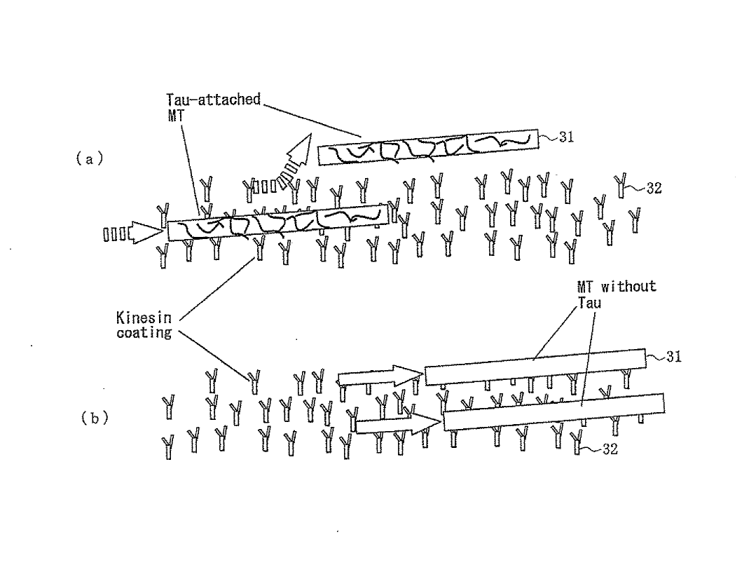

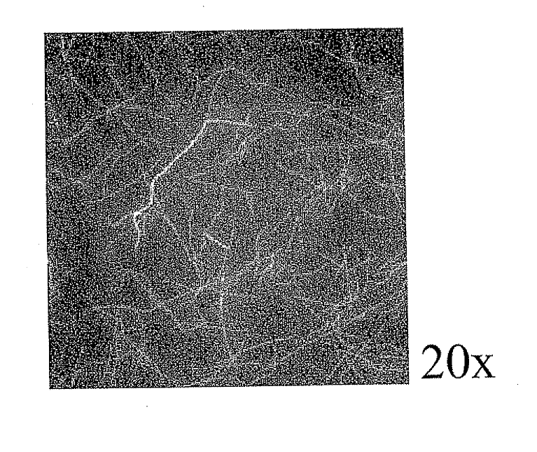

FIG. 14 is a schematic view of Gliding Assay according to the second embodiment of the present invention. FIG. 15 is a set of schematic views of a method of detecting mutated tau according to the second embodiment of the present invention. FIG. 16 is a microphotograph showing microtubules according to the second embodiment of the present invention. In FIGS. 15, (a) shows a disturbed kinesin motion and (b) shows a normal kinesin motion.

In the present embodiment, the second method of detecting protein will be described, and repeated description of matters similar to those of the first embodiment is omitted.

Then, as the second method of detecting protein, only a method for detecting mutated tau will be explained. Specifically, this method, called as “Gliding Assay” or “in-vitro Gliding Assay”, is to detect protein by monitoring microtubules 31 and is a way, with using fluorescent microtubules as the microtubules 31, to judge existence and rate of mutated tau included in a sample by monitoring movements of the fluorescent microtubules along a surface of base 22, made of glass plate and coated with kinesin 32, as shown in FIG. 14. The fluorescent microtubules can be obtained, as the invention described in Patent Document 1, by mixing fluorescent tubulins labeled with fluorescent pigment and tubulins not labeled with fluorescent pigment at appropriate ratio.

The microtubules 31 are stabilized with paclitaxel, as those of the first embodiment. Therefore, in the case tau-protein is attached to the microtubules 31, as shown in FIG. 15(a), motion of kinesin 32 thereon is supposed to be disturbed, resulting in slow motion of kinesin 32 or dropping of molecules of kinesin 32 from the microtubules 31. On the other hand, in the case tau-protein is not attached to the microtubules 31, as shown in FIG. 15(b), motion of kinesin 32 thereon is supposed to be undisturbed, resulting in normal motion of kinesin 32 or rare dropping of molecules of kinesin 32 from the microtubules 31. In other words, it is supposed that, in-vitro, differences would be found in moving distance and moving velocity of the microtubules 31 moving on the base 22 coated with kinesin 32 and in density (or number) of the microtubules 31 on the base 22, according to whether tau-protein is attached or not to the microtubules 31 stabilized with paclitaxel.

Therefore, by feeding a sample including tau-protein purified from a test subject to the microtubules 31 stabilized with paclitaxel and by monitoring motion of the microtubules 31, it would be possible to judge whether the tau-protein included in the sample is healthy or mutated based on the motion of the microtubules 31, and, further, to detect the rate of mutated tau.

Next will be described methods and structures of devices used in the experiments the present inventors conducted.

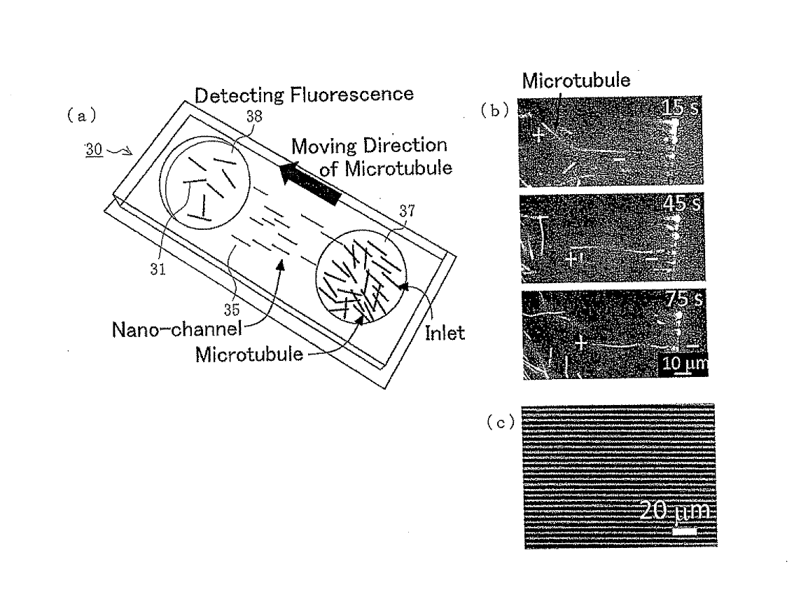

FIG. 17 is a set of views showing devices used in the experiments according to the second embodiment of the present invention. In FIGS. 17, (a) shows a schematic view of the first detecting device, (b) shows a set of photographs showing the state where microtubules move in nano channels and (c) shows a photograph of nano channels.

In the respective figures, a reference numeral 30 designates a second chip, which is a micro-fluidic chip the present inventors produced as a second detection device. The second chip 30 includes a nano channel portion 35, and an inlet 37 and a fluorescence detecting portion 38 as an outlet, both of which are connected to the both ends of the nano channel portion 35.

The nano channel portion 35, produced through photolithography as the nano channel described in Patent Document 1, includes a glass plate and a PDMS film bound to the glass plate, and has a plurality of long and narrow parallel grooves, or nano channels, formed in the PDMS film, as shown in FIG. 17(c). Therefore, the upper surface of the glass plate functions as the bottom surface of each nano channel, and is coated with kinesin 32.

The inlet 37 is a room connected to one end of the nano channel portion 35 and the microtubules 31 diluted in BED 80 buffer solution are fed therein. The microtubules 31 in the inlet 37 are transported by motion of kinesin 32 attached onto the upper surface of the glass plate and, as shown in FIG. 17(b), the microtubules 31 stochastically enter the nano channels. Then, kinesin 32 moves on the microtubule 31 from its minus end toward its plus end, and therefore the microtubule 31 moves in a direction shown by the arrow.

The fluorescence detecting portion 38 is a room connected to the other end of the nano channel portion 35 and the microtubules 31 carried out from the nano channel portion 35 are monitored therein. As mentioned before, differences would be found in moving distance and moving speed of the microtubules 31 transferred by motion of kinesin 32 and in density (or number) of the microtubules 31, according to whether tau-protein is attached or not to the microtubules 31 stabilized with paclitaxel. Therefore, moving distance and moving velocity of the microtubules 31 transferred by motion of kinesin 32 and in density (or number) of the microtubules 31 could be measured by counting the number of the microtubules 81 existing in the fluorescence detecting portion 38 in a predetermined time period after the microtubules 31 are fed in the inlet 37, provided that the length of the nano channel portion 35 and the density (or number) of the microtubules 31 fed in the inlet 37 are given. Further, since intensity of fluorescence in the fluorescence detecting portion 38 is supposed to be proportional to the number of the microtubules 31 therein, moving distance, moving velocity and density (or number) of the microtubules 31 also could be measured by detecting intensity of fluorescence in the fluorescence detecting portion 38.

Next will be described details and results the experiments the present inventors conducted.

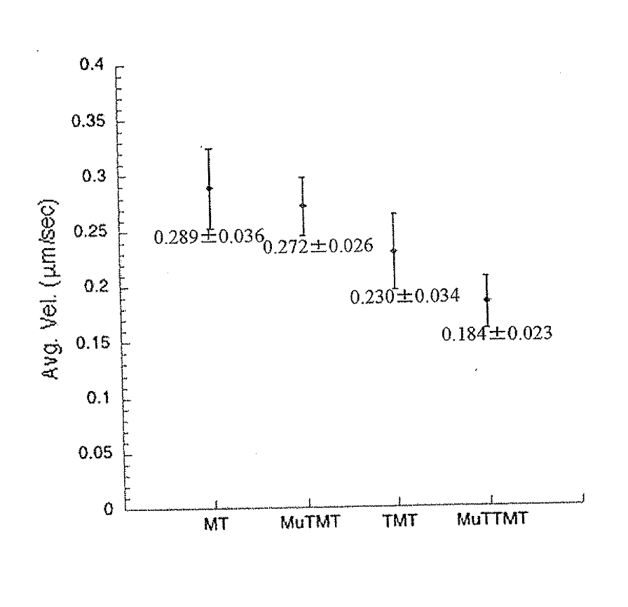

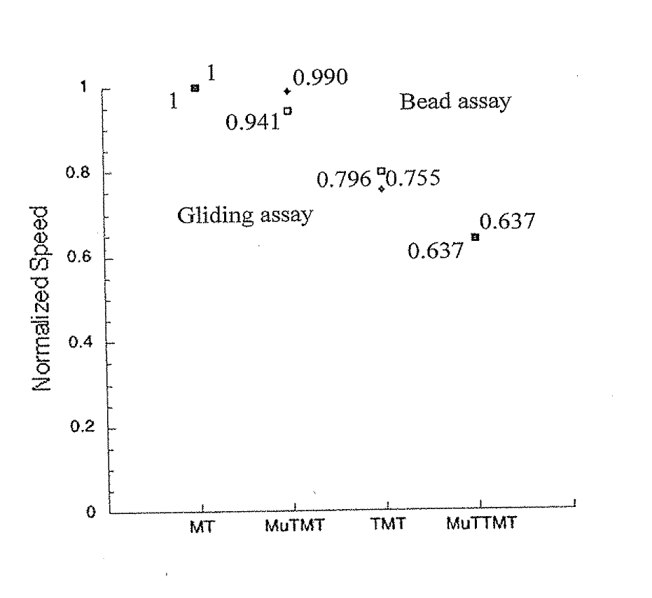

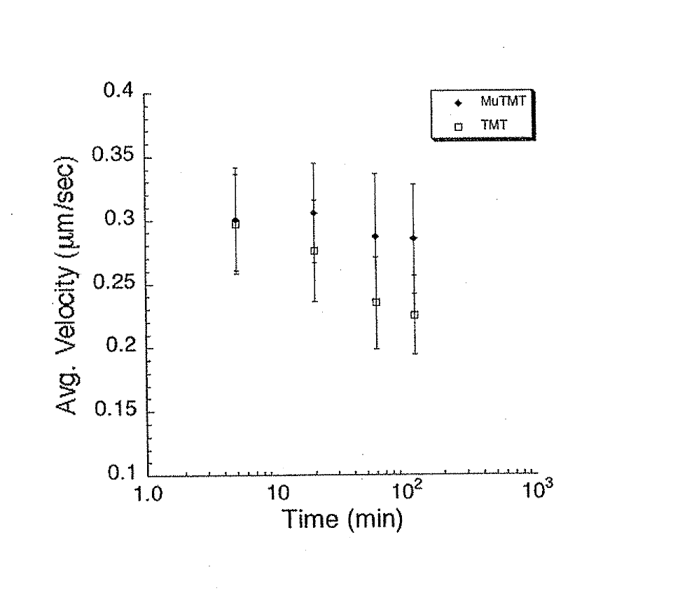

FIG. 18 is a graph showing the effect of tau-protein on motion of kinesin according to the second embodiment of the present invention, FIG. 19 is a graph comparing the motions of kinesin according to the first and second embodiments of the present invention. FIG. 20 is a graph showing the effect of incubation time on motion of kinesin according to the second embodiment of the present invention.

FIG. 18 shows comparison of average velocities of the normal microtubules 31 passing through the nano channel portion 35 with different types of tau attached microtubules 31 in the second chip 30. In FIG. 18, MT designates the case of normal microtubules 31 incubated in BRB 80 buffer solution at 37[° C.] for 20 [min] MuTMT designates the case of mutated tau attached microtubules 31 incubated in BRB 80 buffer solution including mutated tau at 37[° C.] for 20 [min], TMT designates the case of healthy tau attached microtubules 31 incubated in BRB 80 buffer solution including healthy tau at 37[° C.] for 20 [min], and MuTTMT designates the case of healthy tau and mutated tau attached microtubules 31 incubated in BRB 80 buffer solution including healthy tau at 37[° C.] for 20 [min] and incubated in BRB 80 buffer solution including mutated tau at 37[° C.] for 20 [min].

The average velocities of microtubules 31 carried by motion of kinesin 32 in cases of MTs, MuTMTs, TMTs and MuTTMTs were measured as 0.289±0.036 [μm/sec] (n=60), 0.272±0.026 [μm/sec] (n=60), 0.230±0.034 [μm/sec] (n=60), and 0.184±0.023 [μm/sec] (n=60) respectively. The letter n means the number of microtubules 31 monitored.

FIG. 19 shows comparison of the normalized average velocities of microtubules 31 measured by the method called Gliding Assay explained in the present embodiment with the normalized average velocities of beads 36a measured by the method called Bead Assay explained in the first embodiment. In FIG. 19, the symbol “white square” represents the cases of Gliding Assay and the symbol “black diamond” represents the cases of Bead Assay.

As shown in FIG. 19, the ration of the numbers of microtubule 31 and tau-protein stands at 80:1. Further, in oases of Gliding Assay and Bead Assay, the rations of MTs, MuTMTs, TMTs and MuTTMTs stand at 1:0.94:0.80:0.64, and 1:0.99:0.75:0.64.

FIG. 20 shows relation between incubation time and average velocities of microtubules 31. The symbol “white square” represents the cases where microtubules 31 are MTs and the symbol “black diamond” represents the cases where microtubules 31 are TMTs. It is apparent that average velocities of microtubules 31 decreases with increase of incubation time in the cases where microtubules 31 are TMTs.

As described above, in the present embodiment, microtubules 31 are stabilized with paclitaxel, molecules of kinesin 32 are attached on a base 22, a sample including tau-protein is fed to the microtubules 31, and a ratio of mutated tau included in the sample is detected based on motion of microtubules 31 moved by motion of kinesin 32.

And, a second chip 30 includes a nano channel portion 35, an inlet 37 connected to one end of the nano channel portion 35 and a fluorescence detecting portion 38 connected to the other end of the nano channel portion 35, wherein the nano channel portion 35 has a plurality of nano channels with the bottom surface consisted of the top surface of the base 22 where kinesin 32 is attached, microtubules 31 stabilized with paclitaxe are fed in the inlet 37, and the microtubules 31 carried out from the nano channel portion 35 can be monitored in the fluorescence detecting portion 38.

Therefore, in-vitro, the rate of mutated tau included in the sample can be detected easily and surely. Then it would be possible to diagnose tauopathies, typified by Alzheimer, at the stage with low rate of mutated tau, and to cure them early by recovering functions of healthy tau utilizing phosphatase inhibitory agent etc.

The present invention is not limited to the above embodiments, but may be diversely modified and varied, Thus, the modifications and variations are not excluded form the scope of protection oh the present invention.

### INDUSTRIAL APPLICABILITY

The present invention can be applied to a method of detecting protein and a device of detecting protein.

### EXPLANATION OF LETTERS OR NUMERALS

22 base
26 main channel
29a first sub channel
29b second sub channel
35 nano channel portion
36a bead
37 inlet
38 fluorescence detecting portion

1. A method of detecting protein based on a relative motion of a rail molecule which has a polarity, and a biomolecular motor which moves on the rail molecule in a direction corresponding to the polarity of the rail molecule, the method comprising:
(a) stabilizing the rail molecule with stabilizing agent;
(b) attaching either the rail molecule or the biomolecular motor onto a base;
(c) feeding a sample including tau-protein to the rail molecule; and
(d) detecting a ratio of mutated tau-protein included in the sample, based on motion of the biomolecular motor moving on the rail molecule or on motion of the rail molecule moved by action of the biomolecular motor.

2. A method of detecting protein according to claim 1, wherein the ratio of mutated tau-protein included in the sample is detected, based on the fact that motion of the biomolecular motor on the rail molecule stabilized with stabilizing agent is disturbed by healthy tau-protein included in the sample and is not disturbed by mutated tau-protein.

3. A method of detecting protein according to claim 1, wherein the stabilizing agent is paclitaxel or decetaxel.

4. A method of detecting protein according to claim 2, wherein the motion comprises moving velocity, moving distance or density or number of the biomolecular motor moving on the rail molecule, or moving velocity, moving distance or density or number of the rail molecule moved by action of the biomolecular motor.

5. A method of detecting protein according to claim 4, wherein the biomolecular motor binding with a micro-sphere is fed to the rail molecule attached onto a base, so that the motion of the biomolecular motor moving on the rail molecule is judged based on motion of the micro-sphere.

6. A method of detecting protein according to claim 4, wherein the rail molecule with fluorescence is fed to the biomolecular motor attached onto a base, so that the motion of the rail molecule moved by action of the biomolecular motor is judged based on motion of the rail molecule with fluorescence.

7. A detection device used for a method of detecting protein based on a relative motion of a rail molecule which has a polarity, and a biomolecular motor which moves on the rail molecule in a direction corresponding to the polarity of the rail molecule, the detection device comprising:
(a) a main channel having an upper surface of a base as a bottom surface where a rail molecule stabilized with stabilizing agent is attached; and
(b) a sub channel extending in a direction crossing the main channel and being fed with a sample including tau-protein, and wherein
(c) motion of the biomolecular motor moving on the rail molecule can be monitored.

8. A detection device according to claim 7, wherein a plurality of sub channels are included so that effects of tau-protein included in a plurality of samples on motions of the biomolecular motors moving on the rail molecules can be monitored simultaneously.

9. A detection device according to claim 7, wherein the biomolecular motor binding with a micro-sphere is fed in the main channel so that the motion of the biomolecular motor moving on the rail molecule is judged based on motion of the micro-sphere.

10. A detection device used for a method of detecting protein based on a relative motion of a rail molecule which has a polarity, and a biomolecular motor which moves on the rail molecule in a direction corresponding to the polarity of the rail molecule, the detection device comprising:
(a) a nano channel portion including a plurality of nano channels each of which has an upper surface of a base as a bottom surface where a biomolecular motor is attached;
(b) an inlet connected to one end of the nano channel portion so that the rail molecule stabilized with stabilizing agent is fed in the inlet; and
(c) an outlet connected to the other end of the nano channel portion so that the rail molecule fed in the inlet and carried out from the nano channel portion can be detected in the outlet.

11. A detection device according to claim 10, wherein the rail molecule fed in the inlet with fluorescence so that intensity of fluorescence can be detected in the outlet.
